(19) United States
(12) Patent Application Publication
(10) Pub. No.: US 2008/0023014 A1
(43) Pub. Date: Jan. 31, 2008

(54) Device for tuning body to schumann resonance and method of use

(76) Inventor: Jeanne Katherine Gallick, Oroville, CA (US)

Correspondence Address: ERIC HANSCOM, 7395 PORTAGE WAY, CARLSBAD, CA, 92011, US

(21) Appl. No.: 11/478,104

(22) Filed: Jun. 30, 2006

(51) Int. Cl. A61N 1/14; A61N 1/16

(52) U.S. Cl. 128/897; 128/908

(57) ABSTRACT

This invention is directed toward a device which amplifies a user's ground or resonance to the Schumann Resonance, thereby removing a source of distortion and chaos from the user's nervous system caused by AC electrical systems. This increased resonance dissipates stress, makes the user more open to various forms of energy healing. The device relies upon groups of seven or fourteen circuits which amplify the grounding effect, thereby allowing a user of the invention to detoxify himself or herself naturally. Scalar enhancement is also possible when two or more devices, which can be worn as a pendant or plugged into an AC outlet, are used simultaneously.

### CROSS REFERENCE TO RELATED APPLICATIONS

None.

### STATEMENT REGARDING FEDERALLY SPONSORED RESEARCH OR DEVELOPMENT

This invention was not federally sponsored.

### FIELD OF THE INVENTION

This invention relates to the general field of devices to allow a user to ground himself or herself to the electromagnetic field of the earth, and more specifically, to a device which amplifies a user's ground or resonance to the Schumann Resonance, thereby removing a source of distortion and chaos from the user's nervous system caused by AC electrical systems. This increased resonance dissipates stress caused by the generation of biologically meaningless current from the AC electrical system, thus making the user more open to various forms of energy healing. The device relies upon seven imbedded circuits that tune the user's nervous system to the Schumann Resonance and thereby amplify the grounding effect. Scalar enhancement is possible when the seven circuits that are the subject of the patent are matched exactly by another seven circuits. This matching can be created by combining a pendant with a plug in device, each containing seven circuits, or by combining 14 circuits in a pendant or a plug-in device.

### BACKGROUND OF THE INVENTION

History of resonance and the importance of modulation. The invention relates to a discovery made in 1952 by physicist W. O. Schumann, who discovered that the space between the crust of the earth and the ionosphere created a resonating cavity similar to that found in a musical instrument, such as a violin or guitar. Within this cavity, Schumann observed an electromagnetic field (EMF), powered by lightning strikes, which oscillated at a resonant frequency of 7.8-8.0 Hz, which is the exact same frequency at which human brain waves function optimally. This resonating quality became known as the Schumann Resonance.

Resonance is created when one frequency rises, lowers, or comes to rest with another frequency. Once resonance is established, energy can be transferred from one vibrating system to another, as when an opera singer is able to break a glass at a distance when her vibrating voice establishes resonance with the vibrations of the glass. It has been noted that individuals of many different religious backgrounds all had similar alpha brain wave patterns between 7.8 and 8.0 Hz during meditation, which suggests that during meditation the person meditating is resonant to the Schumann Resonance. Therefore, this frequency of vibration appears very strongly to provide optimal awareness and functioning. The concept of modulation is also important to this invention and can be visualized in the following way: imagine music being played into a stream of electrical current with a microphone and then transformed using a vibrating needle into a disk of wax. Another needle can then pick up the vibrations and transform them into another electrical current, which is then demodulated by the vibrating diaphragms of the speakers. In the same way every form, every thought, every action has an impact in the vibrating Schumann Resonance and is broadcast at the speed of light within the entire resonating ionspheric cavity. A human body picks up and responds to this information. On the level of optimal health, people rely upon the Schumann Resonance for many basic biological timing mechanisms and a sense of fundamental wellbeing.

Deleterious effects of AC current and cyclotronic resonance. As a nation, we have chosen alternating current as the means of transporting electrical current (as opposed to DC or direct current). The fundamental law of electricity states that whenever magnetic fields shift back and forth, electrical current is created in any nearby conductor. Therefore, a side effect of the shifting magnetic fields (60 per second in the U.S.) is the generation of biologically meaningless current in the human nervous system. The human nervous system evolved in relationship to the information held in the electromagnetic field of the earth. The AC electrical system that humans have lived with for the past century or so has weakened the human resonance to the earth by inducing chaotic or non-biologicaily meaningful currents into our bodies. These currents interfere with the attunement to nature's magnetic field, thereby overriding many signals humans have evolved to internally rely upon for optimal biological functioning.

Many experiments have been done that indicate high stress levels and significant correlations to rates of cancer created by our AC electrical system. See B. Blake Levitt, Electromagnetic Fields for a summery of these experiments.

Four main explanations have been postulated as to why an AC electrical system has such widespread detrimental effects:

1. The AC grid suppresses the human acupuncture system. Acupuncture meridians are measurable DC (direct current) lines of current that signal where healing is necessary in a human's body. The AC grid common in most households creates a masking effect that suppresses the meridian system and thereby degrades the body's ability to heal itself. By freeing up the meridian system from this chronic stress, the invention supports the body to fully heal itself as is nature's design.

2. The AC grid creates a subliminal state of constant stress that, over time, degrades the immune system. The debilitating effects of this stress on the immune system are well documented. Once the immune system is freed from this constant subliminal stress, through use of the invention, overall health begins to improve as the person is able to fight off infections.

3. The AC electrical system separates humans from the information stored and resonating in the earth's electromagnetic field that allows human bodies to respond to and be regenerated by nature.

4. The concept of Cyclotronic Resonance explains the transference of energy from the electrical system into human cellular metabolism and thus explains three major health crises in the modern world; stress-induced arthritis, the increase in Alzheimer's disease, and chronic depression

Cyclotronic Resonance is discussed in detail in Dr. Robert Becker's book, Cross Currents. It refers to the principle that if two lines of AC current come together in a square grid, as in the wiring of an average home or office, energy can be transferred into a person standing within the grid if the frequencies are close enough. Most electrical systems have a frequency of 60 Hz, which is close enough to the frequency of our biological systems which operate on a frequency of approximately 10 Hz. Energy is transferred from the spinning electrons (charged particles) in the wiring into the spinning ions (electrolytes) in a person's nervous system.

The deleterious biological effects of this energy transference are substantial and explain, in part, the far reaching and yet hidden effects of EMFs on human health. When resonance is established between the house wiring and a human nervous system, spinning ions, such as calcium, potassium, sodium and lithium, which normally govern cell metabolism, are physically ejected from the cell into the bloodstream where they are deposited in the joints or eliminated. Calcium, potassium and lithium are ions that govern calmness, mental clarity, and mood stabilization. Some specific examples are: accumulation of calcium in the joints has been related to arthritis; lack of potassium is related to Alzheimer's disease and the lack of lithium is related to chronic depression and manic behavior.

Thus, it is desirable to reverse the interference caused by our AC electrical system. The invention reduces the effect of EMFs on the nervous system, thereby mitigating cyclotronic resonance and helping the human body to regulate its own healing in a stress reduced environment.

The importance of grounding and geometric circuits.

Grounding is important in two fundamental senses. First, grounding allows for the positive flow of information that connects humans to the ecosystem in which we live. Second, increased grounding also allows the body to rid itself of the biologically meaningless stress inducing currents inadvertently generated in the human nervous system by the shifting magnetic fields of our AC electrical system.

To understand why grounding protects against the hazardous effects of EMFs, it is necessary to discuss how electricity is generated when a magnet is moved back and forth. When this happens a field is created and current is generated in any conductor in the presence of this field. Whether in a nuclear power plant or a coal plant, power is utilized to move a magnet back and forth thereby generating current in coils of wire. In the early 1900's a decision was made to install AC rather than DC current because AC current self-induces itself over long distances, thereby allowing the electricity to be generated far from where it is utilized. Because we have chosen to shift or pulse our current back and forth with alternating current, a side effect is that extraneous biologically meaningless current is generated in our nervous systems whenever we are inside an electrified house.

How it works:

When a person is grounded, this extraneous current will dissipate into the earth, similar to how an electrical circuit often has a ground wire that is connected to a metal spike driven into the ground. One way to ground a person (and thereby re-establish resonance to the earth) is to physically touch the earth. One can also ground an electrical system into the EMF field in which the system is immersed; i.e., the ground can be amplified by increasing the resonance to the earth. The inventor has created a means to amplify grounding through the use of seven resonating geometric circuits that strengthen resonance with the Schumann Resonance and thereby lesson the effects of AC current on the human body. The amplification is accomplished by three geometric circuits that duplicate the geometric structure of the electromagnetic field of the earth. These geometric circuits function as antennae. In the same way that a linear piece of wire resonates to FM wave lengths, so too geometric antennae are shaped as a sphere, a torus, and a spiral to pick up and resonate to the earth's electromagnetic field. The sphere of course resonates to the shape of the earth. The torus relates to the shape of any electromagnetic fields that surround any object that is generating it. In other words, the EMF surrounding the earth is shaped like a torus, a large donut with the north pole of the earth at one end of the hole and the south pole at the other. A similarly shaped torus surrounds the human body with the head at one end of the hole and the feet at the other. The third geometric shape concerns the spiral which is the course of the movement of the earth in space.

The inventor has discovered that resonance or attunement to the Schumann Resonance is increased if the three geometric circuits are constructed of two different kinds of conducting materials. Therefore, the sphere, the torus, and the spiral are constructed of metal and silicon for a total of six circuits, with the seventh circuit a metal container surrounding the six circuits, thereby heightening resonance.

In linear circuits, current is generated and then moves through a wire to do work while on its way to its opposite polarity. In geometric circuits, the generated energy fills a three-dimensional space and vibrates the object. The effect of the vibration of this geometric circuit is to bring a human into greater resonance (allowing the transference of energy and information) with the electromagnetic field surrounding the earth, which contains geometric properties based on a sphere, a torus and a spiral. The discovery of a geometric circuit which creates an increased bond with the earth and conveys a sense of calmness, elation and mental clarity is an important discovery and inventive step. The invention utilizes geometric circuits which are all tuned to the Schumann Resonance as described using difference geometric qualities that underlie the structure of the earth's electromagnetic field, thereby amplifying grounding and allowing greater energy transference. In effect, greater resonance is established with the electromagnetic field of the earth.

Thus there has existed a long-felt need for a natural means to combat the negative effects of AC-created electrical fields in a person's house and around a person. The current invention provides just such a solution by having a device which amplifies a user's ground or resonance to the earth, thereby removing a source of distortion and chaos from the user's nervous system caused by AC electrical systems. This increased resonance dissipates stress, and makes the user more open to various forms of energy healing.

The inventor provides two ways to introduce the circuits into the nervous system of a person. One way is for the person to wear a pendant around the neck (or arm or ankle). George Lakhovsky is the inventor of the spiral oscillating circuit as a healing modality. He describes his invention in a book called The Secret of Life which was published in the 1920s. At first he used a generator to oscillate the copper spiral at a predetermined frequency. Then he discovered that a generator was not necessary, since the circuit oscillated by itself in a way as to restore a diseased plant or person to health. The inventor has duplicated Lakhovsky's oscillating circuit in both metal and silicon, as well as adding two other geometric circuits, the sphere and the torus. The invention also miniaturizes the circuits so that they can be inserted into a resonator or metal container and worn around the neck or other limb as a pendant.

The second method of introducing the circuits into the nervous system is to insert the circuits into the AC wiring of a home or office using a conventional electric plug. No current is actually utilized. The circuits are modulated into the AC current and thereby transferred utilizing the principle of Cyclotronic Resonance into the nervous systems of any human beings within the AC grid of the home. The invention consists of two sets of plug configurations. One type of plug contains seven circuits. The other set of plugs consists of dividing the circuits such that one plug contains three circuits and the other plug contains four circuits to allow for a more gradual introduction of the healing circuits into a person's nervous system.

Besides using circuits as geometric antennae to strengthen resonance to the earth, the device also relies upon the electromagnetic principle of scalar standing waves. A scalar effect occurs when one wave is exactly matched by another wave of the same frequency and amplitude but with are opposite polarity. Therefore the inventor discovered that matching her oscillating circuits with another exact set of seven circuits created a scalar enhancement of the grounding effect. Scalar enhancement occurs when two or more devices, which can be worn as a pendant or plugged into an AC outlet, are used simultaneously. The inventor has also developed a pendant that contains all 14 matched circuits and also a plug in devices that introduces the full 14 matched circuits in three stages. Because some people have damaged immune systems, the inventor has discovered that a three-stage process of introducing the circuits into the person's nervous system minimizes many detoxification symptoms allowing for a more gradual healing process. Therefore, the inventor has developed two types of plug in devices. One contains the seven circuits and the other consists of three plugs that contain three, four and seven circuits each to allow for a graduated introduction of the circuits.

Whether the invention is worn on the body or introduced into the AC system of a home, it strengthens the resonance between the person's nervous system and the earth vibration. The increase in resonance between the human and the earth allows greater energy flow/information as well as the grounding or dissipation of chaotic or AC induced currents into the earth.

With the version that is plugged into the AC outlet the protection offered by the invention will provide protection against the problems presented by EMF's to all humans and animals in the house, apartment, or office in which the one or more units are installed. This protection, caused by the invention's ability to enhance grounding or resonance to the Schumann Resonance, can be magnified due to the scalar enhancement which results if one uses two or more of the AC units at one time, Thus, a user of the invention can protect himself or herself with the pendant version of the invention, and/or all occupants and visitors to a building through use of the AC version of the invention.

### SUMMARY OF THE INVENTION

It is a principal object of the invention to provide a means by which a user can counteract the problems caused by AC current in a house or office.

It is another object of the invention that the device is small, compact, and easily worn on the body or plugged into an electrical outlet.

It is an additional object of the invention that the device can be used all over the world with all currents.

Further objects of the invention include the desire to provide a device by which a person can amplify his/her grounding with the earth.

It is a further object of the invention that the device allows a person to re-establish resonance with the Schumann Resonance.

It is also an object of this invention that by re-establishing resonance with earth, a user can reduce the effect of EMF's on the nervous system, thereby preventing cyclotronic resonance to the AC grid and helping the human body to cure these imbalances.

A further object of the invention is that a user of the invention can protect all occupants of a building against EMF's by using one or more of the AC versions of the invention.

An additional object of the invention is that a user can provide scalar enhancement to all occupants of a building by using two or more of the AC versions of the invention concurrently.

Another object of the invention is to provide a pendant version of the invention which can provide personal protection from EMF's to the wearer of the pendant.

It is a final object of this invention the device be of a small, compact design, and be capable of being manufactured with relatively low cost, thereby translating into an affordable retail price.

It should be understood the while the preferred embodiments of the invention are described in some detail herein, the present disclosure is made by way of example only and that variations and changes thereto are possible without departing from the subject matter coming within the scope of the following claims, and a reasonable equivalency thereof, which claims I regard as my invention.

### BRIEF DESCRIPTION OF THE FIGURES

FIG. 1 is a top view of the invention showing the location of the various components of the invention.

FIG. 2 is a side, perspective view of the invention showing the location of the various components of the invention.

### DETAILED DESCRIPTION OF THE INVENTION

The invention has two basic iterations: a pendant worn around the neck with seven or fourteen circuits, and a unit which plugs into an AC electrical wall unit which has, likewise, seven or fourteen circuits. Both iterations are grounding devices that contain specialized circuits that enable a human to tune his/her body to the Schumann Resonance. These circuits amplify a human's ground in the electromagnetic field of the earth. All circuits within the invention are tuned to the Schumann Resonance and thus allow for a more powerful resonance to be set between the human body and the earth. By amplifying this ground, a channel is opened up that allows for the dissipation of biologically irrelevant currents induced by the AC system to dissipate into the earth. At the same time, this same channel allows for a more intimate bonding to the information held within the earth's electromagnetic field.

Both inventions rely on a series of seven geometric circuits. The seven circuits operate either independently or as a total synergy to amplify grounding. They can be utilized one by one or all together. Furthermore, a scalar enhancement has been discovered by the inventor in which if the seven are exactly matched by another seven such that a metal sphere is matched by a metal sphere, a silicon sphere is matched by a silicon sphere, a metal torus is matched by a metal torus, a silicon torus is matched by a silicon torus, a metal spiral is matched by a metal spiral, a silicon spiral is matched by a silicon spiral, and finally a metal container is matched by a metal container, then scalar resonance is activated. The circuits work together via a synergistic mechanism known as a scalar wave which is a wave that is exactly matched by another wave, i.e. has the same frequency and the same height or amplitude but has the opposite direction, such that one wave exactly cancels out the other. A physical example of this phenomenon would be if two ocean waves of the same frequency and amplitude but coming from different directions crashed into each other and canceled each other out. A biological example of the generation of scalar waves occurs when the left and right sides of the human nervous system are working together in balance. The invention incorporates the principle of scalar enhancement by combining the circuits in particular ways so as to establish a scalar ground or enhancement when any two devices which include the invention are used together. For instance, the iteration which plugs into a wall socket provides basic EMF protection to everyone in the house or office. The neck pendant iteration of the invention provides basic EMF protection to the individual wherever he or she goes. But if a person is wearing a neck pendant iteration inside a house protected by the wall socket iteration, then a scalar enhancement is activated, which provides the maximum EMF protection when the person is in the house or office. Another way to activate maximum EMF protection is to install two of the socket iterations in one house or office, thereby providing everyone within the home with the maximum protection of scalar resonance or to insert 14 circuits into a pendant that provides the wearer with the maximum protection or scalar resonance wherever the user of the invention goes.

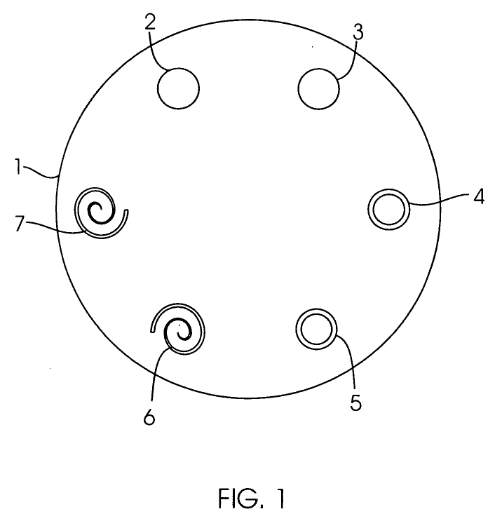

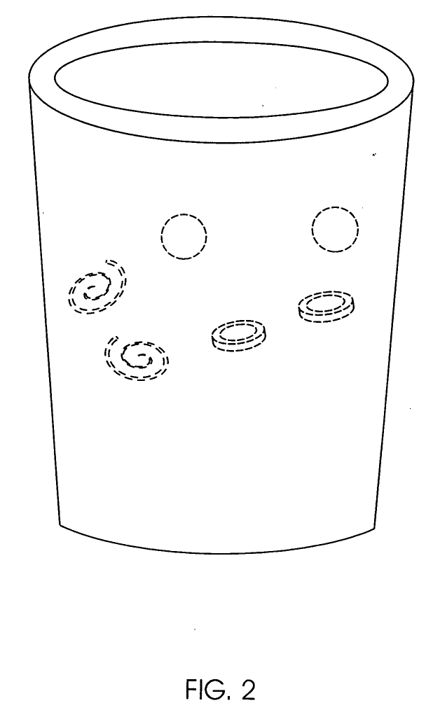

Referring to FIGS. 1 and 2, the invention consists of seven circuits which interrelate to each other to synergistically promote EMF protection for the user of the invention. Turning to FIG. 1, there are six various circuits within an enclosing container (1), which is, preferably, constructed of metal and serves as the seventh circuit. The enclosing container may also be constructed of plastic and covered with aluminum. The circuits are each enclosed in insulating material and placed within the container. While the enclosing container (1) can be rotated about any axis and remain functional, the location and sequence within the enclosing container of the specific circuits is not important. The first figure discussed is a solid silicon sphere (2), clockwise from which can be found, in this order, a solid metal sphere (3), a silicon torus (4), a metal torus (5), a silicon spiral (6), and a metal spiral (7). The metal spiral (7) is also called a Lahkovsky Oscillating Circuit.

Turning to the individual circuits, the silicon sphere (2) and the metal sphere (3) are spherical in shape. The silicon torus (4) is a sphere of silicon with a central hollow core which extends from one end of the sphere to the other, thereby rendering it visible from either end. The metal torus (5) is a sphere of metal which also has a central hollow core which extends from one end of the sphere to the other, thereby rendering it visible from either end. The silicon spiral (6) is a spiral-shaped, flat piece of silicon which is wound, from inside to outside, in a clockwise fashion or counter clockwise fashion before it is inserted into the enclosing container. The metal spiral or Lahkovsky Oscillating Circuit (7) is a piece of stainless steel wire which is wound, from inside to outside, either in a counter-clockwise or counter clockwise fashion. Both the silicon spiral (6) and the metal spiral (7) need only coil once in order to be effective. It is essential that all circuits be encased in insulating material before being inserted into the hollow metal container (1). In the case of the fourteen circuit pendant, a separate hollow metal container must be devised to complete the scalar resonance.

What I claim as my invention is:

1. A device for protecting one or more humans from the deleterious effects of AC current, comprising:
one or more hollow metal containers,
where each of the one or more enclosing metal containers contains one or more circuits within the enclosing container, and
an amount of insulating material sufficient to fill the enclosing container and prevent contact of the circuits with each other within the insulating material, and,
where, each enclosing container is also a circuit, and where, the circuits are tuned to the Schumann Resonance, and where, the circuit or circuits perform a function of enabling a human to tune his/her body to the Schumann Resonance, thereby enabling the human to ground himself or herself and thereby facilitate the healing mechanisms of the human body.

2. The device of claim 1, where there is only one enclosing metal container and the one enclosing container contains at least one circuit and it is selected from a group comprising: a solid silicon sphere, a solid metal sphere, a silicon torus, a metal torus, a silicon spiral and /or a metal spiral.

3. The device of claim 1, where, there is only one enclosing metal container and the one enclosing container contains only two circuits and they are selected from a group comprising: a solid silicon sphere, a solid metal sphere, a silicon torus, a metal torus, a silicon spiral and/or a metal spiral.

4. The device of claim 1, where, there is only one enclosing metal container and the one enclosing container contains three circuits and they are selected from a group comprising: a solid silicon sphere, a solid metal sphere, a silicon torus, a metal torus, a silicon spiral and/or a metal spiral.

5. The device of claim 1, where, there is only one metal enclosing container and the one enclosing container contains four circuits and they are selected from a group comprising: a solid silicon sphere, a solid metal sphere; a silicon torus, a metal torus, a silicon spiral and/or a metal spiral.

6. The device of claim 1, where, there is only one metal enclosing container and the one enclosing container contains five circuits and they are selected from a group comprising: a solid silicon sphere, a solid metal sphere, a silicon torus, a metal torus, a silicon spiral and/or a metal spiral

7. The device of claim 1, where, there is only one enclosing metal container and the one enclosing container contains six circuits and they are selected from a group comprising: a solid silicon sphere, a solid metal sphere, a silicon torus, a metal torus, a silicon spiral and/or a metal spiral.

8. The device of claim 1, where the device comprises two enclosing metal containers and one enclosing metal container contains one circuit to six circuits and it is selected from a group comprising: a solid silicon sphere, a solid metal sphere, a silicon torus, a metal torus, a silicon spiral and/or a metal spiral.

9. The device of claim 1, where the device is a pendant version of the invention in which the enclosing metal container is less than 4 inches long and less than 1 inch in diameter, and where the device additionally comprises a means of attachment on the side of the enclosing container which will allow the device to be attached by a chain or other flexible means of attachment around a user's neck such that the device can be worn as a pendant.

10. The device of claim 1, where the device is an A/C wall outlet version of the invention in which the device additionally comprises means of plugging the device into a common A/C wall outlet, where the enclosing metal container containing one to thirteen circuits, each insulated from each other, can be inserted into a common electrical plug.

11. The device of claim 1, additionally comprising: six circuits, where the six circuits consist of and a solid silicon sphere, a solid metal sphere, a silicon torus, a metal torus, a silicon spiral, and a metal spiral, an amount of insulating material sufficient to fill the enclosing container and prevent movement of the circuits within the insulating material, where, the enclosing container is also a circuit, and where, the circuits are tuned to the Schumann Resonance, and where, the circuits, synergistically perform a function of enabling a human to tune his/her body to the Schumann Resonance.

12. The device of claim 11, where the circuits amplify a human's ground in the electromagnetic field of the earth, thereby allowing for a more powerful resonance to be set between the human body and the earth, allowing for the dissipation of biologically irrelevant currents induced by the AC system into the earth.

13. The device of claim 1, additionally consisting of: an enclosing metal container which functions as a circuit and encloses at least one circuit of six specified circuits, where the six circuits consist of a solid silicon sphere, a solid metal sphere, a silicon torus, a metal torus, a silicon spiral, and a metal spiral, an amount of insulating material sufficient to fill the enclosing container and prevent movement of the circuits within the insulating material, where, and where, the enclosing container is also a circuit, and where, the circuits are tuned to the Schumann Resonance, and where, the circuits, synergistically perform a function of enabling a human to tune his/her body to the Schumann Resonance.

14. The device of claim 13, where the circuits amplify a human's ground in the electromagnetic field of the earth, thereby allowing for a more powerful resonance to be set between the human body and the earth, allowing for the dissipation of biologically irrelevant currents induced by the AC system into the earth.

15. A method for protecting one or more humans from the deleterious effects of AC current, comprising:
a first step of obtaining one or more devices, where each device comprises an enclosing container which allow a user to ground himself or herself with the electromagnetic field of the earth, and more specifically, to a device with one or more circuits tuned to the Schumann Resonance, and where, the circuit or circuits synergistically perform a function of enabling a human to tune his/her body to the Schumann Resonance,
a second step of attaching the device or devices to a human body or A/C outlet,
and a third step of allowing the device to synergistically perform a function of enabling a human to tune his/her body to the Schumann Resonance, thereby enabling the human to detoxify himself or herself.

16. The method of claim 15, where, in the first step the user obtains a pendant version of the device and an A/C wall outlet version of the device and uses both the pendant version and the A/C wall outlet version simultaneously such that scalar enhancement occurs.

17. The method of claim 15, where, in the first step the user obtains two A/C wall outlet versions of the device and uses both A/C wall outlet versions simultaneously such that scalar enhancement occurs.

18. The method of claim 15, where, in the first step the user obtains a two pendant versions of the device and uses both pendant versions simultaneously such that scalar enhancement occurs.

19. The method of claim 15, where, in the first step the user obtains more than two devices, where the devices are selected from the group consisting of the pendant version and the A/C wall outlet version.

20. The method of claim 15, where, in the first step the enclosing container contains two sets of seven circuits, where there are two enclosing containers and inside of each enclosing container are six circuits.
